(19) United States
(12) Patent Application Publication
(10) Pub. No.: US 2019/0167232 A1
(43) Pub. Date: Jun. 6, 2019

(54) PLANE WAVE ULTRASOUND IMAGING

(71) Applicants: Bahador Makkiabadi, Tehran (IR); Moein Mozaffarzadeh, Ramsar (IR); Seyed Hani Hozhabr, Tehran (IR); Ali Ghasemi, Tehran (IR); Fatemeh Mehrzad, Tehran (IR)

(72) Inventors: Bahador Makkiabadi, Tehran (IR); Moein Mozaffarzadeh, Ramsar (IR); Seyed Hani Hozhabr, Tehran (IR); Ali Ghasemi, Tehran (IR); Fatemeh Mehrzad, Tehran (IR)

(73) Assignee: Bahador Makkiabadi, Tehran (IR)

(21) Appl. No.: 16/268,531

(22) Filed: Feb. 6, 2019

Related U.S. Application Data
(60) Provisional application No. 62/627,214, filed on Feb. 7, 2018.

(51) Int. Cl. A61B 8/00; A61B 8/14

(52) U.S. Cl. CPC A61B 8/4461; A61B 8/4488; A61B 8/14

(57) ABSTRACT

A method for plane wave (PW) ultrasound imaging is disclosed. The method includes transmitting a PW of a plurality of PWs at a transmission angle from a plurality of ultrasound transducers to a target, receiving a radio frequency (RF) echoes subset of a plurality of RF echoes at a non-zero reception angle by the plurality of ultrasound transducers, generating a migrated image of a plurality of migrated images from the RF echoes subset by applying a migration method on the RF echoes subset, and generating an ultrasound image of the target from the migrated image.

### CROSS-REFERENCE TO RELATED APPLICATION

This application claims the benefit of priority from pending U.S. Provisional Patent Application Ser. No. 62/627,214, filed on Feb. 7, 2018, and entitled “AN ULTRASOUND IMAGING SYSTEM,” which is incorporated herein by reference in its entirety.

### TECHNICAL FIELD

The present disclosure generally relates to medical imaging, and particularly, to ultrasound imaging.

### BACKGROUND

Ultrasound imaging (USI) is a common method in medical imaging. Image quality this imaging modality may be concerned with the amount of presence of off-axis signals and artifacts in acquired data. In B-mode USI, a target of imaging may be sequentially swept by multiple lines of a transmitted signal and received signals may be used to form an ultrasound (US) image.

The number of lines used for sweeping an imaging medium and the depth of imaging may affect the frame rate (FR) of USI. Sound velocity may be another factor of FR limitation in B-mode USI. On the other hand, there are some applications, such as 3-D USI and echocardiography, which may require a high FR. To this end, there have been a number of investigations to increase FR of USI. One of the solutions is USI in plane wave (PW) mode, where the FR is not impacted by the depth of imaging. However, a high FR in PW USI may be obtained at a cost of having a low image quality. There is, therefore, a need for a plane Wave ultrasound imaging method that provides a high image quality.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for plane wave (PW) ultrasound imaging. An exemplary method may include transmitting a PW of a plurality of PWs at a transmission angle from a plurality of ultrasound transducers to a target, receiving a radio frequency (RF) echoes subset of a plurality of RF echoes at a non-zero reception angle by the plurality of ultrasound transducers, generating a migrated image of a plurality of migrated images from the RF echoes subset by applying a migration method on the RF echoes subset, and generating an ultrasound image of the target from the migrated image.

In an exemplary embodiment, transmitting the PW may include generating the PW by exciting the plurality of ultrasound transducers, and steering the PW to the transmission angle. In an exemplary embodiment, receiving the RF echoes subset may include steering the RF echoes subset to the non-zero reception angle. In an exemplary embodiment steering the RF echoes subset to the non zero reception angle comprises steering the RF echoes subset to the transmission angle.

In an exemplary embodiment, applying the migration method on the RF echoes subset may include applying a frequency-wavenumber (f-k) migration method on the RF echoes subset. In an exemplary embodiment, applying the f-k migration method on the RF echoes subset may include applying a Stolt's f-k migration method on the RF echoes subset.

In an exemplary embodiment, generating the ultrasound image may include extracting an envelope of the migrated image. In an exemplary embodiment, generating the ultrasound image may include generating a plurality of aligned images by aligning the plurality of migrated images, generating an averaged image by weighted averaging the plurality of aligned images, and generating the ultrasound image by extracting an envelope of the averaged image. In an exemplary embodiment, aligning the plurality of migrated images may include rotating each of the plurality of migrated images from the non-zero reception angle to a zero angle.

In an exemplary embodiment, generating the ultrasound image may include generating a plurality of aligned images by aligning the plurality of migrated images, generating a plurality of envelope images by extracting an envelope of each of the plurality of aligned images, and generating the ultrasound image by weighted averaging the plurality of envelope images.

In an exemplary embodiment, receiving the RF echoes subset may include sequentially receiving portions of the RF echoes subset by a plurality of reception sub-arrays, and integrating the received portions of the RF echoes subset into a single image. Each of the plurality of reception sub-arrays may include a transducers subset of the plurality of ultrasound transducers.

In an exemplary embodiment, transmitting the PW may include sequentially transmitting portions of the PW by a plurality of transmission sub-arrays. Each of the plurality, of transmission sub-arrays may include a transducers subset of the plurality of ultrasound transducers. In an exemplary embodiment, receiving the RF echoes subset may include sequentially receiving portions of the RF echoes subset by a plurality of reception sub-arrays, and integrating the received portions of the RF echoes subset into a single image. A reception sub-array of the plurality of reception sub-arrays may include a segment of a transmission sub-array of the plurality of transmission sub-arrays. In addition, a center of the reception sub-array may coincide with a center of the transmission sub-array.

In an exemplary embodiment, the present disclosure describes an exemplary system for PW ultrasound imaging. An exemplary system may include a transducer array, a transmit beamformer, a memory having processor-readable instructions stored therein, and one or more processors. In an exemplary embodiment, the transducer array may be configured to transmit a PW of a plurality of PWs at a transmission angle from the transducer array to a target, and receive a radio frequency (RF) echoes subset of a plurality of RF echoes at a non-zero reception angle. In an exemplary embodiment, the transmit beamformer may be configured to generate the PW by exciting a plurality of ultrasound transducers of the transducer array, and steer the PW to the transmission angle. In an exemplary embodiment, the one or more processors ma be configured to access the memory and execute the processor-readable instructions, which, when executed by the one or more processors may configure the one or more processors to perform an exemplary method. An exemplary method may include steering the RF echoes subset to the non-zero reception angle, generating a migrated image of a plurality of migrated images from the RF echoes subset by applying a migration method on the RF echoes subset, and generating an ultrasound image of the target from the migrated image. In an exemplary embodiment, the non-zero reception angle may equal the transmission angle.

In an exemplary embodiment, the transmit beamformer may be further configured to sequentially excite a plurality of transmission sub-arrays via a multiplexer. Each of the plurality of transmission sub-arrays may include a transducers subset of the plurality of ultrasound transducers.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for plane wave ultrasound imaging, consistent with one or more exemplary embodiments of the present disclosure.

FIG. 1B shows a flowchart of transmitting a plane wave, consistent with one or more exemplary embodiments of the present disclosure.

FIG. 1C shows a flowchart of receiving a radio frequency echoes subset, consistent with one or more exemplary embodiments of the present disclosure.

FIG. 1D shows a flowchart of generating an ultrasound image, consistent with one or more exemplary embodiments of the present disclosure.

FIG. 1E shows a flowchart of generating an ultrasound image, consistent with one or more exemplary embodiments of the present disclosure.

FIG. 2 shows a plane wave transmitted from a plurality of ultrasound transducers, consistent with one or more exemplary embodiments of the present disclosure.

FIG. 3A shows a sequence of receiving a radio frequency echoes subset, consistent with one or more exemplary embodiments of the present disclosure.

FIG. 3B shows a sequence of transmitting and receiving a radio frequency echoes subset, consistent with one or more exemplary embodiments of the present disclosure.

FIG. 4 shows a schematic of a system for plane wave ultrasound imaging, consistent with one or more exemplary embodiments of the present disclosure.

FIG. 5 shows a high-level functional block diagram of a computer system, consistent with one or more exemplary embodiments of the present disclosure.

FIG. 6A shows a reconstructed ultrasound image of a complicated wire-target phantom in a sector of about 3 degrees, consistent with an exemplary embodiment of the present disclosure.

FIG. 6B shows a reconstructed ultrasound image of a complicated wire-target phantom in a sector of about 6 degrees, consistent with an exemplary embodiment of the present disclosure.

FIG. 6C shows a reconstructed ultrasound image of a complicated wire-target phantom in a sector of about 9 degrees, consistent with an exemplary embodiment of the present disclosure.

FIG. 7 shows lateral variations of reconstruct ultrasound images, consistent with an exemplary embodiment of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method and system for plane wave ultrasound imaging. An exemplary method aims ID improve the imaging quality by sending and receiving plane waves from an ultrasound transducer array with non-zero transmission and reception angles. For this purpose, transmitted waves may be steered to a desired direction (angle) by utilizing a beamformer and received echoes may be virtually steered to a desired direction by applying a virtual time delay to each signal that is received by a transducer element. The received signals may be processed by a migration method (i.e., rearranged in a transformed image) to obtain an ultrasound image of a target. To decrease the imaging cost, the transmission and reception processes may be sequentially performed by utilizing a portion of elements of the transducer array and integrating the received images into a single image, so that less hardware may be required for processing the signals at each step of transmission/reception.

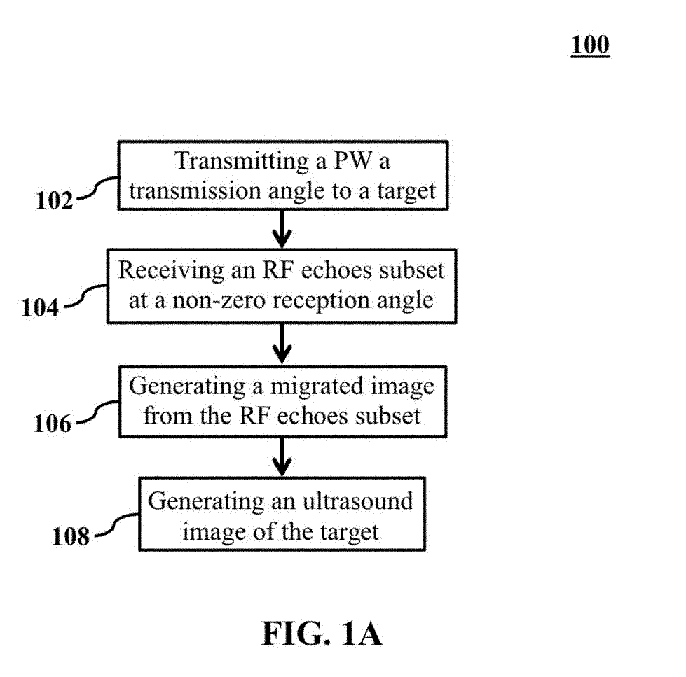

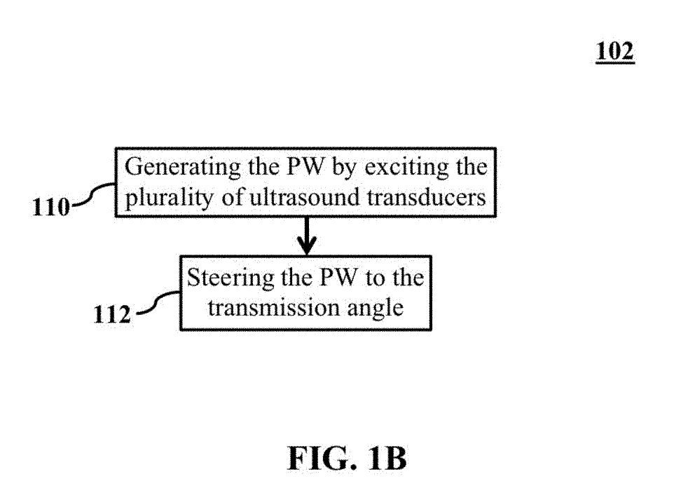

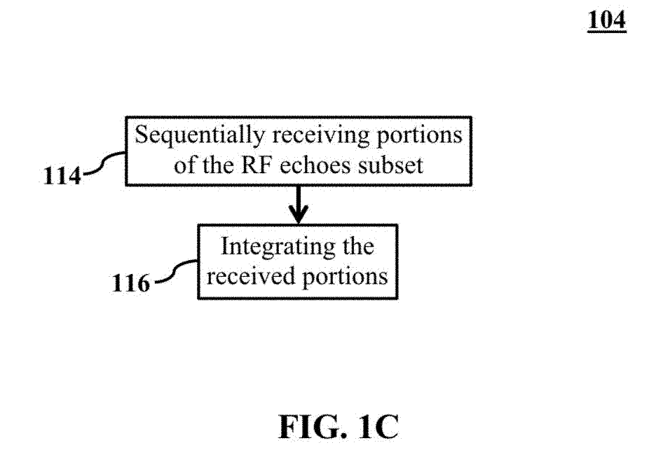

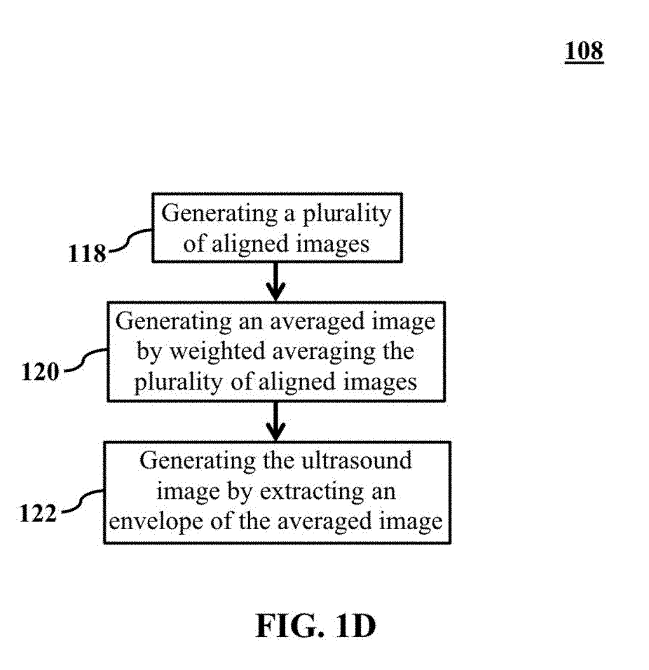

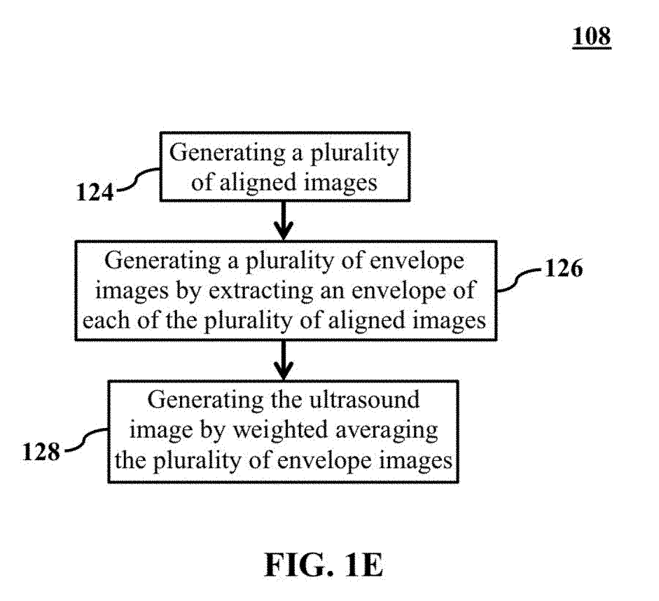

FIG. 1A shows a flowchart of a method for plane wave (PW) ultrasound imaging, consistent with one or more exemplary embodiments of the present disclosure. An exemplary method 100 may include transmitting a PW of a plurality of PWs at a transmission angle from a plurality of ultrasound transducers to a target (step 102), receiving a radio frequency (RF) echoes subset of a plurality of RF echoes at a non-zero reception angle by the plurality of ultrasound transducers (step 104), generating a migrated image of a plurality of migrated images from the RF echoes subset by applying a migration method on the RF echoes subset (step 106), and generating an ultrasound image of the target from the migrated image (step 108).

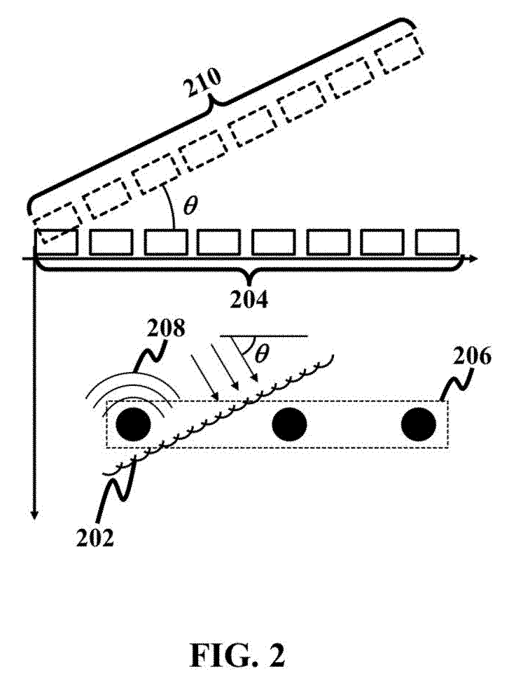

For further detail with regards to method 100, FIG. 2 shows a PW 202 transmitted from a plurality of ultrasound transducers 204 consistent with one or more exemplary embodiments of the present disclosure. In an exemplary embodiment, PW 202 may be transmitted from plurality of ultrasound transducers 204 to a target 206 at a transmission angle θ. In an exemplary embodiment, target 206 may include a number of scatterers that may reflect or diffract PW 202 to different directions. Reflected RF echoes 208 from target 206 may be received by plurality of ultrasound transducers 204.

For further detail with respect to step 102, FIG. 1B shows a flowchart of transmitting PW 202, consistent with one or more exemplary embodiments of the present disclosure. Referring to FIGS. 1A, 1B, and 2, in an exemplary embodiment, transmitting PW 202 (step 102) may include generating PW 202 by exciting plurality of ultrasound transducers 204 (step 110) and steering PW 202 to transmission angle θ (step 112). In an exemplary embodiment, a beamformer may be utilized to excite plurality of ultrasound transducers 204 by sending an electric pulse to plurality of ultrasound transducers 204. In an exemplary embodiment, the beamformer may perform the excitation (step 110) for different transducer elements with different time delays so that the resulting PW may be steered to transmission angle θ (step 112). In other words, by applying different time delays to different transducer elements of plurality of ultrasound transducers 204, a virtual plurality of ultrasound transducers 210 may be implemented by virtually steering plurality of ultrasound transducers 204 to transmission angle θ, which may transmit PW 202 at transmission angle θ.

The cost of ultrasound imaging may be reduced by reducing the number of elements contributed in reception. Therefore, in an exemplary embodiment, ultrasound echoes may be received by sub-arrays and active sub-arrays may be sequentially shifted to cover an entire imaging area.

In further detail with regards to step 104, FIG. 1C shows a flowchart of receiving the RF echoes subset, consistent with one or more exemplary embodiments of the present disclosure. In an exemplary embodiment, receiving the RF echoes subset (step 104) may include sequentially receiving portions of the RF echoes subset by a plurality of reception sub-arrays (step 114) and integrating the received portions of the RF echoes subset into a single image (step 116). Each of the plurality of reception sub-arrays may include a transducers subset of plurality of ultrasound transducers 204.

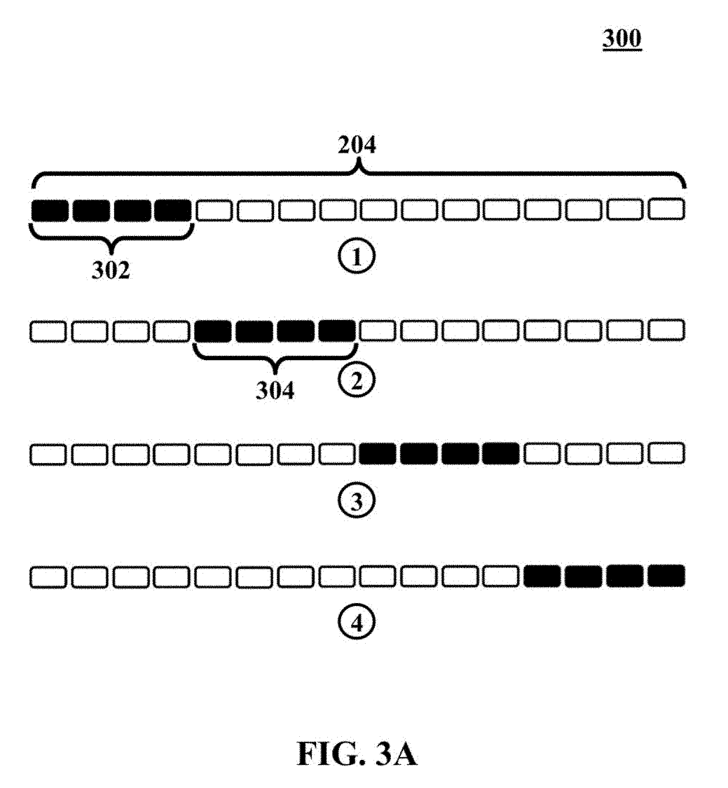

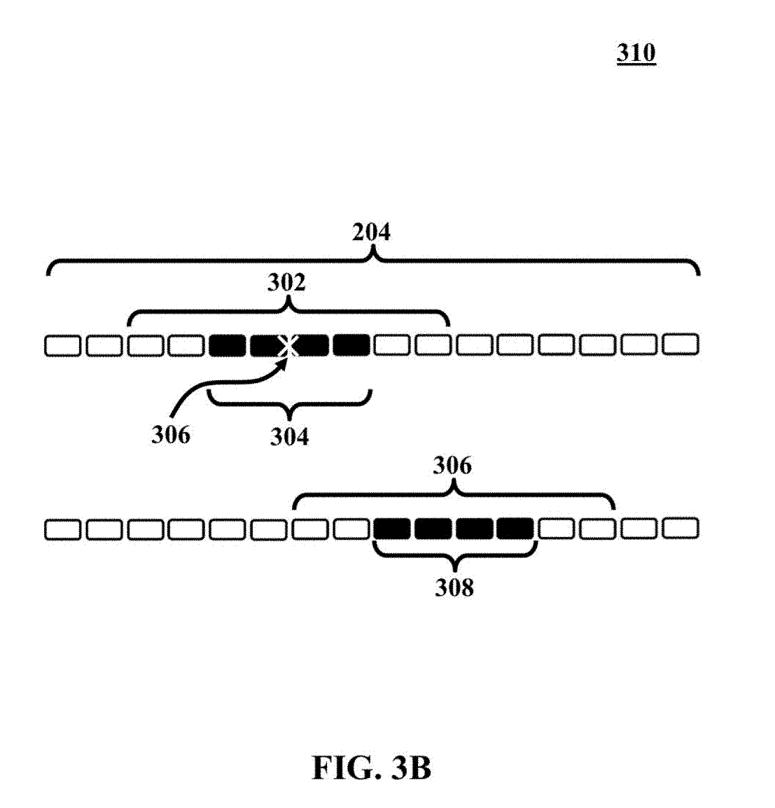

For further detail with respect to step 114, FIG. 3A shows a sequence 300 of receiving the RF echoes subset, consistent with one or more exemplary embodiments of the present disclosure. In an exemplary embodiment, after transmitting PW 202 to target 206, a first portion the RF echoes subset may be received by a reception sub-array 302 of the plurality of reception sub-arrays (shown by black rectangles in FIG. 3A) at step 1 of sequence 300. In an exemplary embodiment, a second portion of the RF echoes subset may be received at step 2 of sequence 300 by a reception sub-array 304. In an exemplary embodiment, this process may be continued until a final portion of the RF echoes subset is received by a last reception sub-array of the plurality of reception sub-arrays at a final step of sequence 300 (for example, step 4 in FIG. 3A). In an exemplary embodiment, each of the plurality of reception sub-arrays, for example, reception sub-array 302 and reception sub-array 304, may include a transducers subset of plurality of ultrasound transducers 204.

The cost of ultrasound imaging may be further reduced by reducing the number of elements contributed in both transmission and reception. Therefore, in an exemplary embodiment, ultrasound echoes may be sent and received by sub-arrays, and active sub-arrays may be sequentially shifted to cover an entire imaging area.

In further detail with respect to steps 102 and 104, FIG. 3B shows a sequence 310 of transmitting and receiving the RF echoes subset, consistent with one or more exemplary embodiments of the present disclosure. In an exemplary embodiment, transmitting PW 202 (step 102) may include sequentially transmitting portions of PW 202 by a plurality of transmission sub-arrays. Each of the plurality of transmission sub-arrays, for example, transmission sub-array 302, may include a transducers subset of plurality of ultrasound transducers 204. In an exemplary embodiment, receiving the RF echoes subset (step 104) may include sequentially receiving portions of the RF echoes subset by a plurality of reception sub-arrays and integrating the received portions of the RF echoes subset into a single image. An exemplary reception sub-array 304 of the plurality of reception sub-arrays may include a segment of a transmission sub-array 302 of the plurality of transmission sub-arrays. In addition, a center 306 of reception sub-array 304 may coincide with a center of transmission sub-array 302. In an exemplary embodiment, after transmitting a portion PW 202 at a step of sequence 310 by transmission sub-array 302, the corresponding portion of the RF echoes subset may be received by reception sub-may 304. In an exemplary embodiment, a next step of sequence 310 may include transmitting a next portion of PW 202 by a transmission sub-array 306 of the plurality of transmission sub-arrays, followed by receiving the corresponding portion of the RF echoes subset by a reception sub-array 308 of the plurality of reception sub-arrays. In an exemplary embodiment, this process may be repeated for all of the plurality of transmission sub-arrays.

For further detail with regards to step 104, receiving the RF echoes subset may include steering the RF echoes subset to the non-zero reception angle. In an exemplary embodiment, steering the RF echoes subset may be perfumed by applying a virtual time delay to each RF signal that may be received by a transducer element of plurality of ultrasound transducers 204. As a result, the RF echoes subset may be received by virtually rotated plurality of ultrasound transducers 204, which may be rotated by the non-zero reception angle. In an exemplary embodiment, the non-zero reception angle, may be set equal to the transmission angle.

In further detail with respect to step 106, the migration method may include rearrangement of the data of the RF echoes subset so that information of true locations of the scatterers in target 206 may be restored in the migrated image. Examples of the migration method include time-domain migration methods, such as the delay and sum (DAS) algorithm, and frequency domain migration methods, such as the finite difference migration algorithm. In an exemplary embodiment, applying the migration method on the RF echoes subset may include applying a frequency-wavenumber (f-k) migration method on the RF echoes subset. Examples of the f-k migration method include the phase-shift migration method and the Stolt's f-k migration method. In an exemplary embodiment, applying the f-k migration method on the RF echoes subset may include applying the Stolt's f-k migration method on the RF echoes subset. In an exemplary embodiment, generating the ultrasound image (step 108) may include extracting an envelope of the migrated image. In an exemplary embodiment, the envelope signal may be obtained by calculating the Hilbert transform of the migrated image.

For further detail with regards to step 108, FIG. 1D shows a flowchart of generating the ultrasound image, consistent with one or more exemplary embodiments of the present disclosure. In an exemplary embodiment, generating the ultrasound image (step 108) may include generating a plurality of aligned images by aligning the plurality of migrated images (step 118), generating an averaged image by weighted averaging the plurality of aligned images (step 120), and generating the ultrasound image by extracting an envelope of the averaged image (step 122).

In an exemplary embodiment, aligning the plurality of migrated images in step 118 may include rotating each of the plurality of migrated images from the non-zero reception angle to a zero angle. In an exemplary embodiment, rotating each of the plurality of migrated images may be performed by multiplying each of the plurality of migrated images by a rotation matrix in which a rotation angle may be set to −α, where α is the non-zero reception angle. In an exemplary embodiment, weighted averaging die plurality of aligned images in step 120 may include weighted averaging corresponding image samples, i.e., pixels, of a plurality of aligned images by a weighted averaging method, such as low pass filtering, median filtering, etc. In an exemplary embodiment, extracting the envelope of the averaged image in step 122 may include calculating a Hilbert transform of the averaged image.

In further detail with regards to step 108, FIG. 1E shows another flowchart of generating the ultrasound image, consistent with one or more exemplary embodiments of the present disclosure. In an exemplary embodiment, generating the ultrasound image (step 108) may include generating a plurality of aligned images by aligning the plurality of migrated images (step 124), generating a plurality of envelope images by extracting an envelope of each of the plurality of aligned images (step 126), and generating the ultrasound image by weighted averaging the plurality of envelope images (step 128).

In an exemplary embodiment, aligning the plurality of migrated images in step 124 may include rotating each of the plurality of migrated images from the non-zero reception angle to a zero angle. In an exemplary embodiment, step 124 may be similar to step 118. In an exemplary embodiment, rotating each of the plurality of migrated images may be performed by multiplying each of the plurality of migrated images by a rotation matrix in which a rotation angle may be set to −α, where α is the non-zero reception angle. In an exemplary embodiment, extracting an envelope of each of the plurality of aligned images in step 126 may include calculating the Hilbert transform of each of the plurality of aligned images. In an exemplary embodiment, weighted averaging the plurality of envelope images in step 128 may include weighted averaging corresponding image samples, i.e., pixels, of the plurality of envelope images by a weighted averaging method, such as low pass filtering, median filtering, etc.

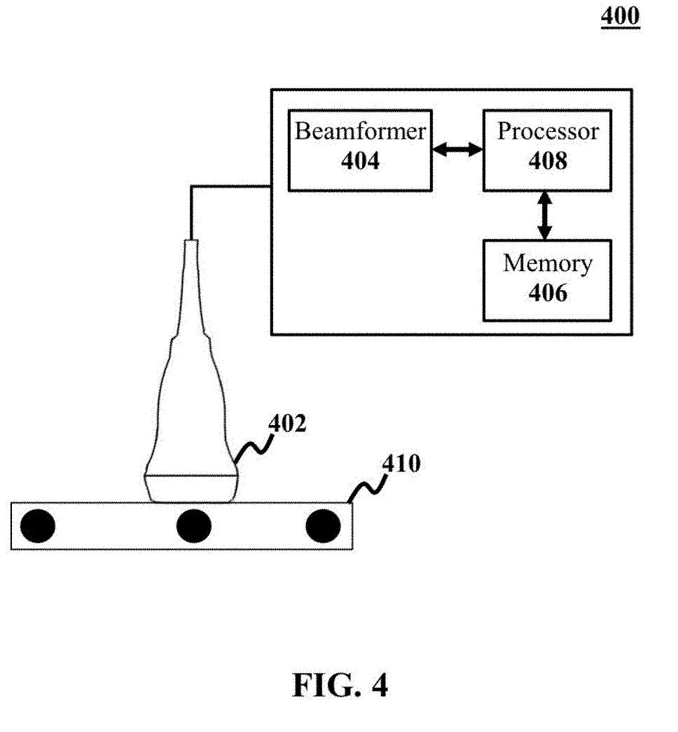

FIG. 4 shows a schematic of a system for PW ultrasound imaging, consistent with one or more exemplary embodiments of the present disclosure. Referring to FIGS. 1A-4, in an exemplary embodiment, different steps of method 100 may be implemented by utilizing an exemplary system 400. An exemplary system 400 may include a transducer array 402, a transmit beamformer 404, a memory 406 having processor-readable instructions stored therein, and one or more processors 408. In an exemplary embodiment, transducer array 402 may be configured to transmit a PW of a plurality of PWs at a transmission angle from transducer array 402 to a target 410 (similar to step 102), and receive an RF echoes subset of a plurality of RF echoes at a non-zero reception angle (similar to step 104). In an exemplary embodiment, transmit beamformer 404 may be configured to generate the PW by exciting a plurality of ultrasound transducers of the transducer array (similar to step 110) and steer the PW to a transmission angle (similar to step 112). In an exemplary embodiment, processor 408 may be configured to access the memory and execute the processor-readable instructions, which, when executed by processor 408 may configure processor 408 to perform an exemplary method. An exemplary method may include steering the RF echoes subset to the non-zero reception angle, generating a migrated image of a plurality of migrated images from the RF echoes subset by applying a migration method on the RF echoes subset (similar to step 106), and generating an ultrasound image of the target from the migrated image (similar to step 108). In an exemplary embodiment, the non-zero reception angle may be equal to the transmission angle.

In an exemplary embodiment, transmit beamformer 404 may be further configured to sequentially excite a plurality of transmission sub-arrays via a multiplexer. Each of the plurality of transmission sub-arrays may include a transducers subset of the plurality of ultrasound transducers.

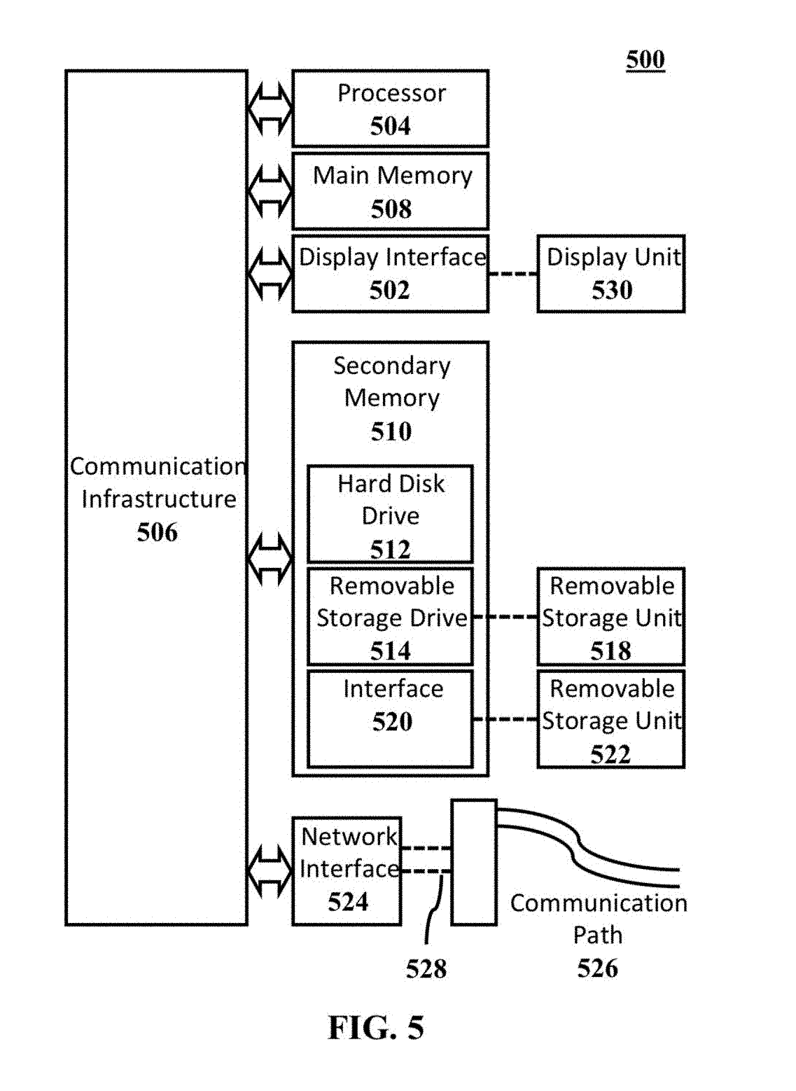

FIG. 5 shows an example computer system 500 in which an embodiment of the present invention, or portions thereof, may be implemented as computer-readable code, consistent with exemplary embodiments of the present disclosure. For example, method 100 may be implemented in computer system 500 using hardware, software, firmware, tangible computer readable media having instructions stored thereon, or a combination thereof and may be implemented in one or more computer systems or other processing systems. In an exemplary embodiment, system 500 may be analogous to processor 408. Hardware, software, or any combination of such may embody any of the modules and components in FIGS. 1A-4.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 500. After reading this description, it will become apparent to a person skilled in the relevant art how to implement the invention using other computer systems and/or computer architectures. Although operations may he described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a duster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in method 100 illustrated by flowcharts of FIG. 1A-FIG. 1E discussed above. Accordingly, such computer programs represent controllers of computer system 500. Where an exemplary embodiment of method 100 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### EXAMPLE

In this example, an exemplary implementation of plane wave (PW) ultrasound imaging is demonstrated. An array of transducers with 128 elements and a pitch of about 290 μm is simulated. Sub-arrays with 48 elements are used for transmission and sub-arrays with 16 elements are used for reception, with an overlap of 8 elements in subsequent sub-arrays for successive transmission/reception processes. Two cycles of a windowed sinusoidal wave at the central frequency of about 5 MHz and a fractional bandwidth of about 80% are utilized in transmission. Noise is added to received ultrasound signals to obtain an SNR of about 40 dB.

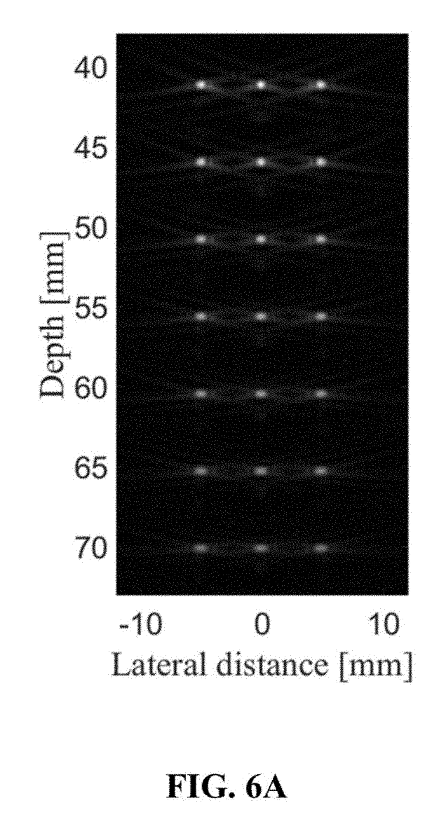

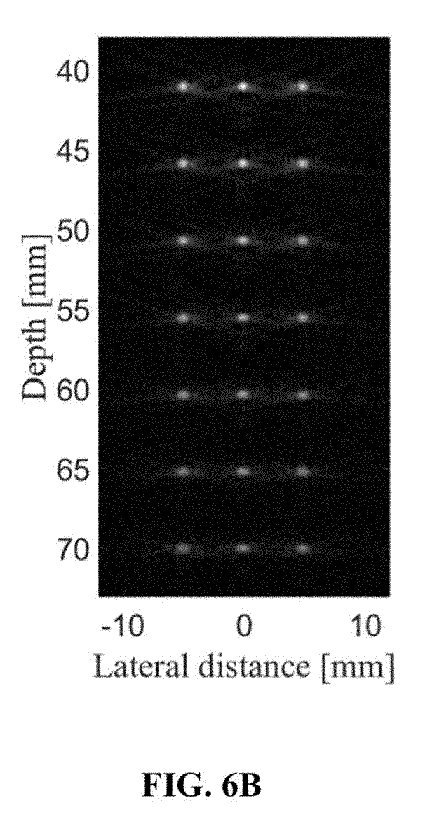

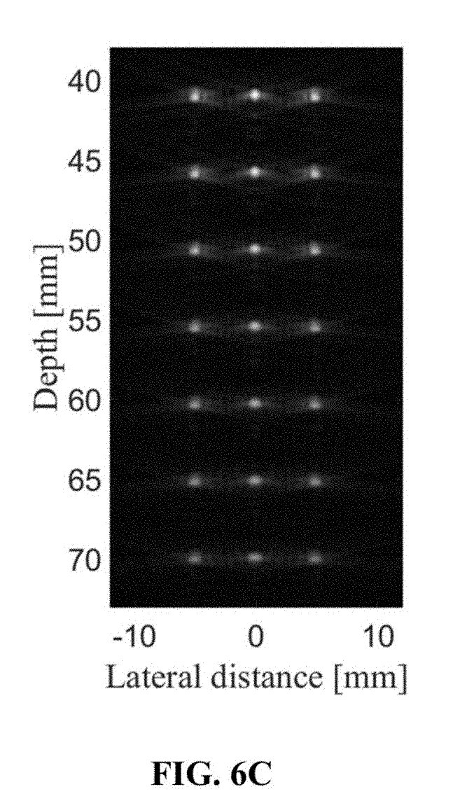

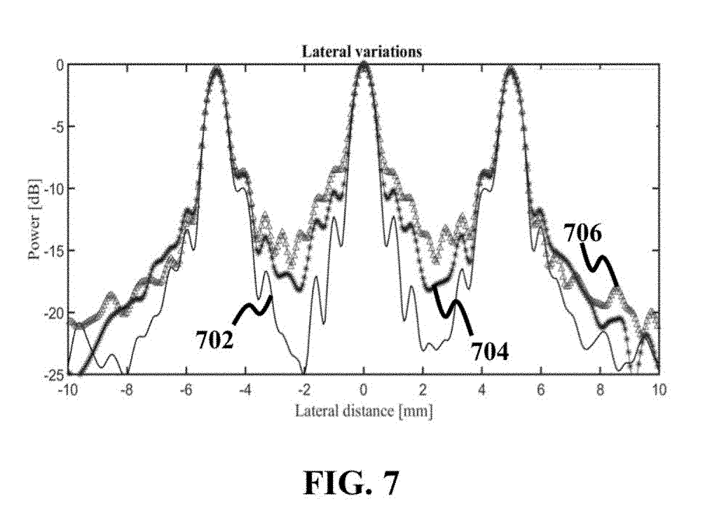

FIG. 6A shows a reconstructed ultrasound image of a complicated wire-target phantom in a sector of about 3 degrees, consistent with an exemplary embodiment of the present disclosure. FIG. 6B shows a reconstructed ultrasound image of a complicated wire-target phantom in a sector of about 6 degrees, consistent with an exemplary embodiment of the present disclosure. FIG. 6C shows a reconstructed ultrasound image of a complicated wire-target target phantom in a sector of about 9 degrees, consistent with an exemplary embodiment of the present disclosure. FIG. 7 shows lateral variations of the reconstructed ultrasound images, consistent with an exemplary embodiment of the present disclosure. Lateral variations 702 of the 3 degrees sector image, lateral variations 704 of the 6 degrees sector image, and lateral variations 706 of the 9 degrees sector image are shown in FIG. 7. It can be observed that sidelobes of the lateral variations increase with the sector size. For example, lateral variations 702 have the smallest sidelobes, whereas lateral variations 706 have the largest sidelobes.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter les in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light the attached claims and their equivalents. Also, various modifications and changes may be made within the scope of the attached claims.

What is claimed is:

1. A method for plane wave ultrasound imaging, the method comprising:
transmitting a plane wave of a plurality of plane waves at a transmission angle from a plurality of ultrasound transducers to a target;
receiving a radio frequency (RF) echoes subset of a plurality of RF echoes at a non-zero reception angle by the plurality of ultrasound transducers;
generating a migrated image of a plurality of migrated images from the RF echoes subset by applying a migration method on the RF echoes subset; and
generating an ultrasound image of the target from the migrated image.

2. The method of claim 1, wherein transmitting the plane wave comprises:
generating the plane wave by exciting the plurality of ultrasound transducers; and
steering the plane wave to the transmission angle.

3. The method of claim 1, wherein receiving the RF echoes subset comprises steering the RF echoes subset to the non-zero reception angle.

4. The method of claim 3, wherein steering the RF echoes subset to the non-zero reception angle comprises steering the RF echoes subset equivalent to an angle of the transmission angle.

5. The method of claim 1, wherein applying the migration method on the RF echoes subset comprises applying a frequency-wavenumber (f-k) migration method on the RC echoes subset.

6. The method of claim 5, wherein applying the f-k migration method on the RF echoes subset comprises applying a Stolt's f-k migration method on the RF echoes subset.

7. The method of claim 1, wherein generating the ultrasound image comprises extracting an envelope of the migrated image.

8. The method of claim 1, wherein generating the ultrasound image comprises:
generating a plurality of aligned images by aligning the plurality of migrated images;
generating an averaged image by weighted averaging the plurality of aligned images; and
generating the ultrasound image by extracting an envelope of the averaged image.

9. The method of claim 8, wherein aligning the plurality of migrated images comprises rotating each of the plurality of migrated images from the non-zero reception angle to a zero angle.

10. The method of claim 1, wherein generating the ultrasound image comprises:
generating a plurality of aligned images by aligning the plurality of migrated images;
generating a plurality of envelope images by extracting an envelope of each of the plurality of aligned images; and
generating the ultrasound image by weighted averaging the plurality of envelope images.

11. The method of claim 1, wherein receiving the RF echoes subset comprises:
sequentially receiving portions of the RF echoes subset by a plurality of reception sub-arrays, each of the plurality of reception sub-arrays comprising a transducers subset of the plurality of ultrasound transducers; and
integrating the received portions of the RF echoes subset into a single image.

12. The method of claim 1, wherein transmitting the plane wave comprises sequentially transmitting portions of the PW by a plurality of transmission sub-arrays, each of the plurality of transmission sub-arrays comprising a transducers subset of the plurality of ultrasound transducers.

13. The method of claim 12, wherein receiving the RF echoes subset comprises:
sequentially receiving portions of the RF echoes subset by a plurality of reception sub-arrays, a reception sub-array of the plurality of reception sub-arrays comprising a segment of a transmission sub-array of the plurality of transmission sub-arrays, a center of the reception sub-array coinciding with a center of the transmission sub-array; and
integrating the received portions of the RF echoes subset into a single image.

14. A system for plane wave ultrasound imaging, the system comprising:
a transducer array comprising a plurality of transducers, the transducer array configured to:
transmit a plane wave of a plurality of plane waves at a transmission angle from the transducer array to a target; and
receive a radio frequency (RF) echoes subset of a plurality of RF echoes at a non-zero reception angle;
a transmit beamformer configured to:
generate the plane wave by exciting the plurality of ultrasound transducers; and
steer the plane wave to the transmission angle;
a memory having processor-readable instructions stored therein; and
one or more processors configured to access the memory and execute the processor-readable instructions, which, when executed by the one or more processors configures the one or more processors to perform a method, the method comprising:
steering the RF echoes subset to the non-zero reception angle;
generating a migrated image of a plurality of migrated images from the RF echoes subset by applying a migration method on the RF echoes subset; and
generating an ultrasound image of the target from the migrated image.

15. The system of claim 14, wherein the non-zero reception angle equals the transmission angle.

16. The system of claim 14, wherein the method further comprises:
applying a Stolt's f-k migration method on the RF echoes subset; and
extracting an envelope of the migrated image.

17. The system of claim 14, wherein the method further comprises:
generating a plurality of aligned images by rotating each of the plurality of migrated images from the non-zero reception angle to a zero angle;
generating a plurality of envelope images by extracting an envelope of each of the plurality of aligned images; and
generating the ultrasound image by weighted averaging the plurality of envelope images.

18. The system of claim 14, wherein the method further comprises:
sequentially receiving portions of the RF echoes subset from a plurality of reception sub-arrays, each of the plurality of reception sub-arrays comprising a transducers subset of the plurality of ultrasound transducers; and
integrating the received portions of the RF echoes subset into a single image.

19. The system of claim 14, wherein the transmit beamformer is further configured to:
sequentially excite a plurality of transmission sub-arrays via a multiplexer, each of the plurality of transmission sub-arrays comprising a transducers subset of the plurality of ultrasound transducers.

20. The system of claim 19, wherein the method further comprises:
sequentially receiving portions of the RF echoes subset from a plurality of reception sub-arrays, a reception sub-array of the plurality of reception sub-arrays comprising a segment of a transmission sub-array of the plurality of transmission sub-arrays, a center of the reception sub-array coinciding with a center of the transmission sub-array; and
integrating the received portions of the RF echoes subset into a single image.
